(19) United States
(12) Patent Application Publication
(10) Pub. No.: US 2011/0305603 A1
(43) Pub. Date: Dec. 15, 2011

(54) Embedded Photocatalyst for Hydrogen Perioxide Protection

(75) Inventor: Timothy J. Flick, Moorhead, MN (US)

(73) Assignee: SIGMA PRIME SOLAR LLC, Fargo, ND (US)

(21) Appl. No.: 12/814,238

(22) Filed: Jun. 11, 2010

(51) Int. Cl. B01J 19/08; B01J 8/00

(52) U.S. Cl. 422/186.3; 422/232; 422/211

(57) ABSTRACT

Embodiments of the present invention combine a suitable photocatalyst with a non-conducting matrix such as plastic or rubber for the purpose of the production of hydrogen peroxide in the presence of light of a suitable frequency or frequencies and oxygenated, acidic water. A suitable photocatalyst such as Anatase titanium dioxide is combined at low temperature (>˜700 F) with a plastic such as polypropylene as one would a pigment. The impregnated plastic can be immersed in water to about an inch whereupon the excess hydrogen ion in the water combines with dissolved oxygen to produce hydrogen peroxide upon irradiation. Hydrogen peroxide is a excellent oxidizer and disinfectant and purifier and goes on to kill bacteria, algae, etc. in the water, as well as to precipitate hardness. Unused hydrogen peroxide breaks down into hydrogen ion and free oxygen in a short time.

### I. FIELD OF THE INVENTION

Embodiments of the present invention generally relates to substantially cleaning impure water. Particularly, embodiments of the present invention relate to a disinfecting apparatus. More particularly embodiments of the present invention relate a disinfectant system for the efficient disinfection of contaminated water.

### II. BACKGROUND OF THE INVENTION

Contaminants within fluid sources (e.g., both air and liquid state) and surfaces are prevalent and can cause great amounts of harm to those animals or plants in contact with the fluid sources. Various types of disinfectants and filtering devices have been used in the past to try and rid the fluid sources of the contaminants.

However, those disinfectants and filtering devices generally do not work properly by not ridding the fluid source of the contaminants and adding further pollutants to the fluid source. This can be very time consuming requiring constant attention, or simply too costly for small production facilities or reservoir structures, such as livestock water tanks, water bottles or toilets.

There have been methods suggested for the use of titanium dioxide in the Anatase form for use in ceramics for producing self disinfecting surfaces. The main drawback is the high working temperatures for ceramic substrates. These would require acidic water to work properly as well. There have also been reported, plastics with antibodies engineered into their matrix to produce antibacterial surfaces, but the process is expensive and selective for certain microorganisms.

Because of the inherent problems with the related art, there is a need for a new and improved disinfectant system for the efficient disinfection of contaminated surfaces and fluids. It would be desirable to find a water purification system where no fossil fuel is needed for sustained operations; disinfection and softening of questionable drinking water is provided; the system is gravity fed requiring no pumps; there are no residual carcinogenic, otherwise toxic or ecologically harmful by products, precise monitoring can be possible thus giving the ability to adjust for the amount of hardness in the feed water and the water has a pleasant taste.

### III. BRIEF SUMMARY OF THE INVENTION

Embodiments of the present invention combine a suitable photocatalyst with a non-conducting matrix such as plastic or rubber for the purpose of the production of hydrogen peroxide in the presence of light of a suitable frequency or frequencies and oxygenated, acidic water. A suitable photocatalyst such as Anatase titanium dioxide is combined at low temperature (>˜700 F) with a plastic such as polypropylene as one would a pigment. The impregnated plastic can be immersed in water to about an inch whereupon the excess hydrogen ion in the water combines with dissolved oxygen to produce hydrogen peroxide upon irradiation. Hydrogen peroxide is a excellent oxidizer and disinfectant and purifier and goes on to kill bacteria, algae, etc. in the water, as well as to precipitate hardness. Unused hydrogen peroxide breaks down into hydrogen ion and free oxygen in a short time.

### IV. BRIEF DESCRIPTION OF THE DRAWINGS

FIG. 1 is an upper perspective view of an embodiment of the present invention within a reservoir structure comprised of a livestock water tank;

FIG. 2 is an upper perspective view of an embodiment of the present invention;

FIG. 3 is an upper perspective view of an embodiment the present invention with the float exploded outwards;

FIG. 4 is a top view of an embodiment of the present invention;

FIG. 5 is a. side sectional view taken along lines 5-5 of FIG. 4;

FIG. 6 is a side cross-sectional view of an embodiment of the present invention within a liquid fluid source;

FIG. 7 is an illustrative cross-sectional view of the carrier showing the photocatalyst evenly distributed throughout the substrate in an embodiment of the present invention;

FIG. 8 is an illustrative cross-sectional view of the carrier showing the treatment surface continually exposed to an outside of the carrier as the carrier degrades during in an embodiment of the present invention;

FIG. 9 is an upper perspective view of the structure comprised of a livestock water tank functions in an embodiment of the present invention;

FIG. 10 is a side view of the carrier within water bottle in an embodiment of the present invention;

FIG. 11 is a top sectional view in an embodiment of the present invention;

FIG. 12 is a top view of the carrier within urinal in an embodiment of the present invention;

FIG. 13 is an upper perspective view in an embodiment of the present invention;

FIG. 14 is an upper perspective view of an embodiment of the present invention;

FIG. 15 is an upper perspective view of embodiments of the present invention positioned over an oil spill on the ground surface;

FIG. 16 is an upper perspective view of embodiments of the present invention;

FIG. 17 is an front profile view of an embodiment in the present invention for a purification system for unclean water;

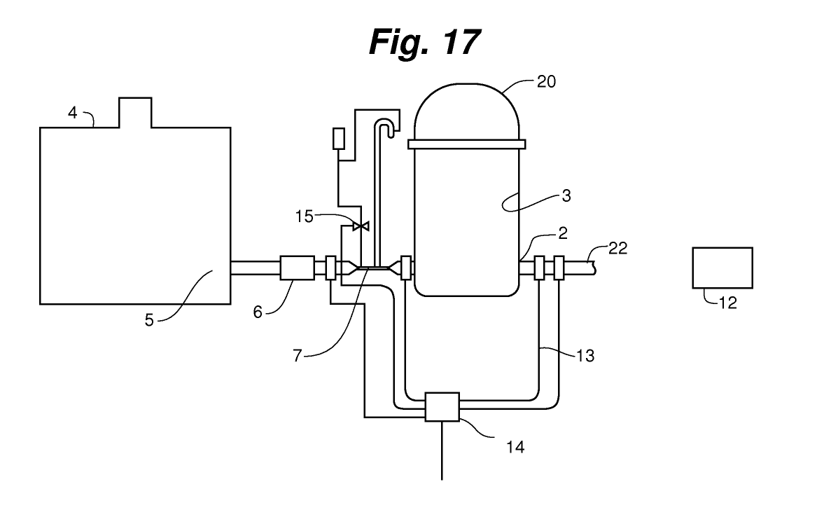

FIG. 18 is a front profile view of an embodiment for an injector system in the present invention; and

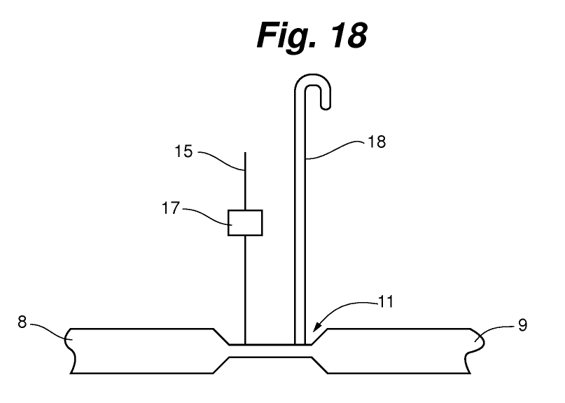

FIG. 19 is a front profile view of a citric acid dispenser in and embodiment of the present invention.

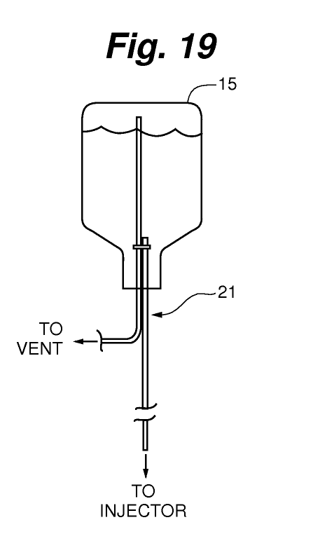

### V. DETAILED DESCRIPTION

The following discussion is presented to enable a person skilled in the art to make and use the present teachings. Various modifications to the illustrated embodiments will be readily apparent to those skilled in the art, and the generic principles herein may be applied to other embodiments and applications without departing from the present teachings. Thus, the present teachings are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of the present teachings. Skilled artisans will recognize the examples provided herein have many useful alternatives and fall within the scope of the present teachings.

Embodiments of the present invention disclose a system for the efficient disinfection of contaminated surfaces and fluids. Embodiments of the present invention generally relate to a disinfecting apparatus which includes a light source for producing ultraviolet light, a fluid source having a mass of organic contaminants within and a carrier comprising a substrate and a photocatalyst. The photocatalyst is evenly distributed throughout the substrate so a treatment surface of the carrier is continually exposed to the fluid source and the ultraviolet light as the substrate degrades. The substrate is comprised of an electrically non conductive material. The treatment surface is positioned at least partially within the fluid source and wherein the ultraviolet light is focused upon the treatment surface for oxidizing the mass of organic contaminants within the fluid source. The carrier may be used for various purposes such as for disinfecting drinking water, ground surfaces, and table surfaces. The carrier may also be supported in various frames or support structures.

The inventor was performing experiments on an inexpensive production method for the production of hydrogen peroxide when it became apparent hydrogen peroxide would be a good disinfection method for producing potable water. Embodiments of the present invention involve the use of very inexpensive ingredients to produce a high cost to benefit ratio. It involves relatively low temperature production methods allowing titanium dioxide to remain in the Anatase form throughout the production process. It also allows for an extended working lifetime since the photocatalyst can be distributed throughout the matrix. As the matrix surface is sloughed off; new catalyst is exposed.

Embodiments of the invention comprise a float attached to the center of either a square or circular flat plastic backing and impregnated grid assembly, or a flat plastic impregnated matrix without backing These units are central to support accessories such as acidifiers, tanks, filters, plumbing and sensors with controller(s).

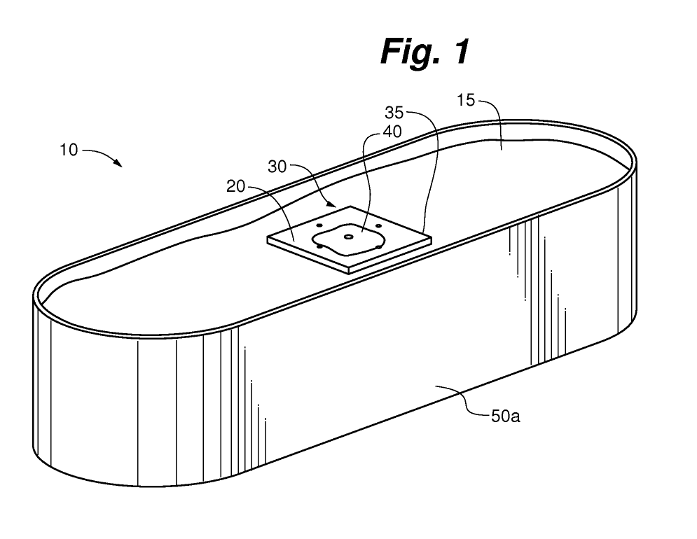

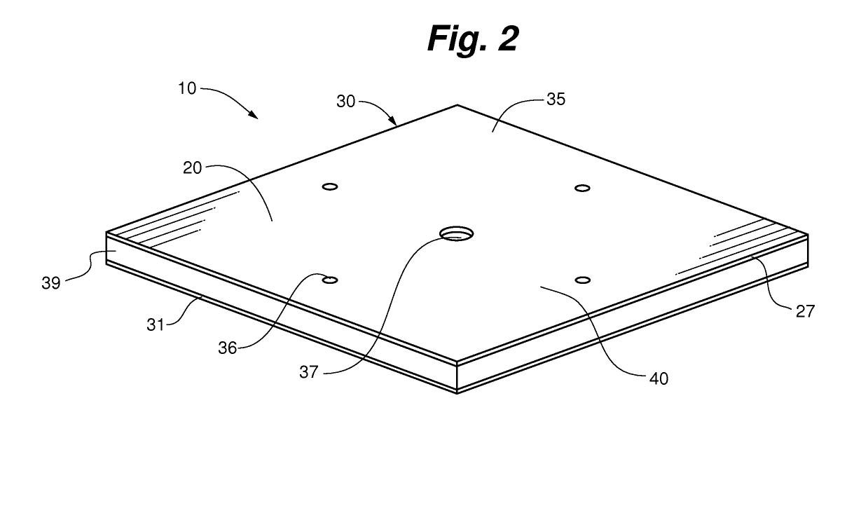

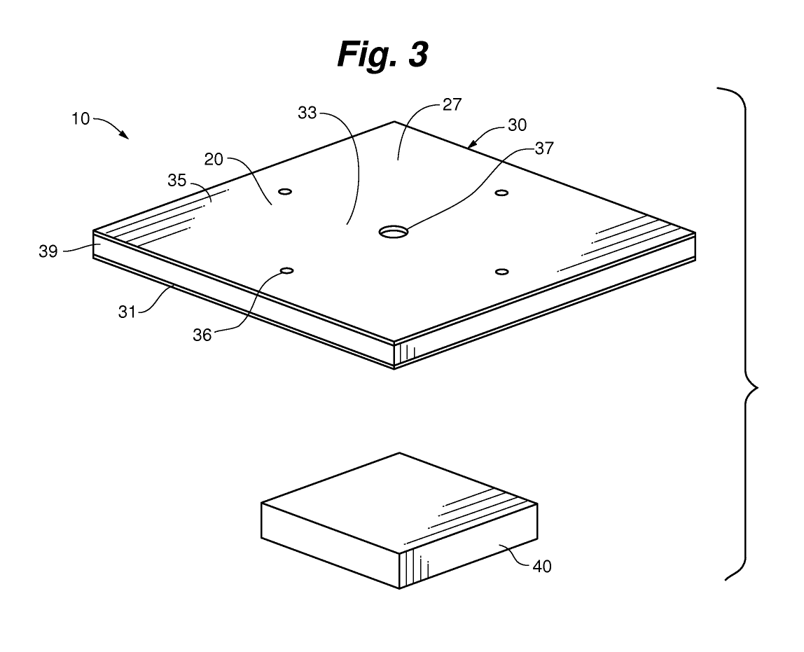

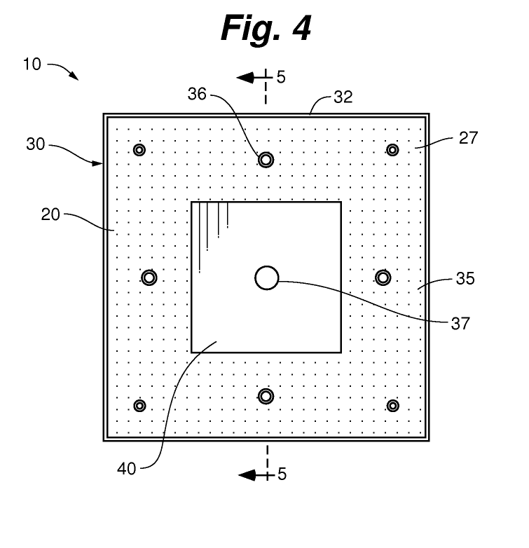

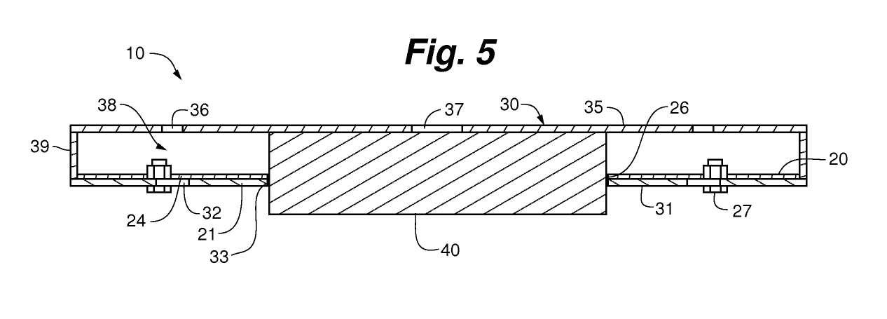

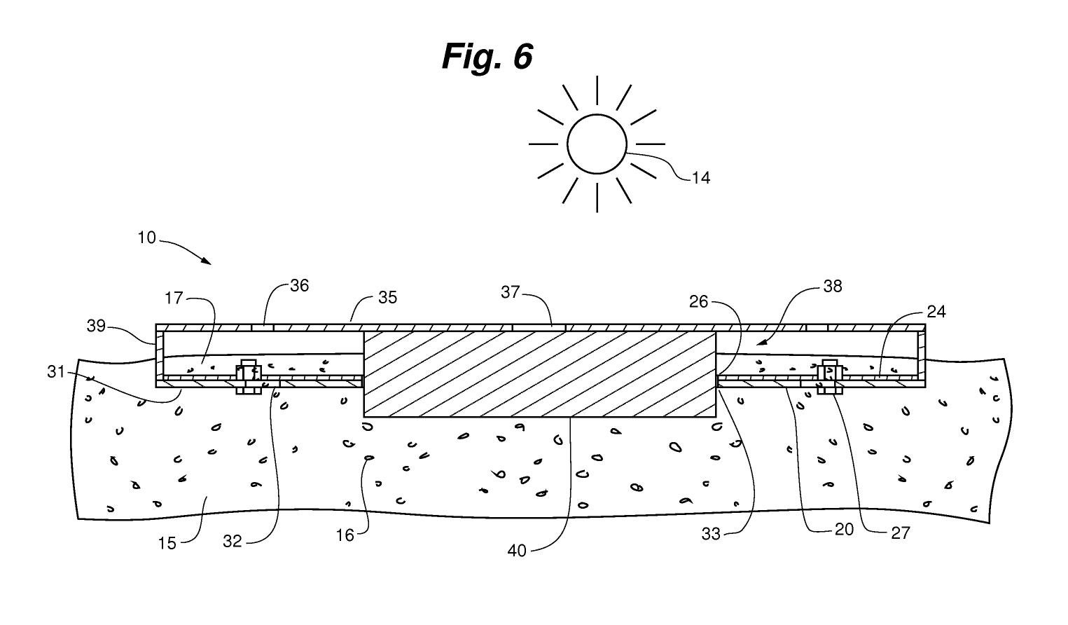

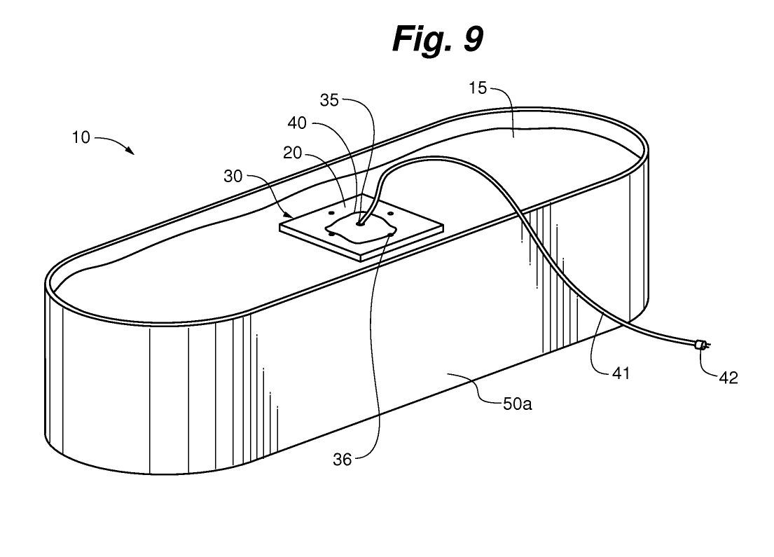

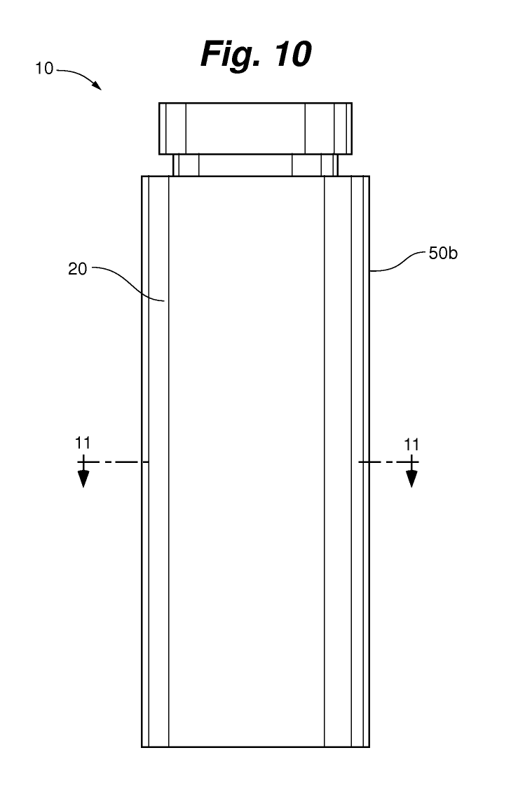

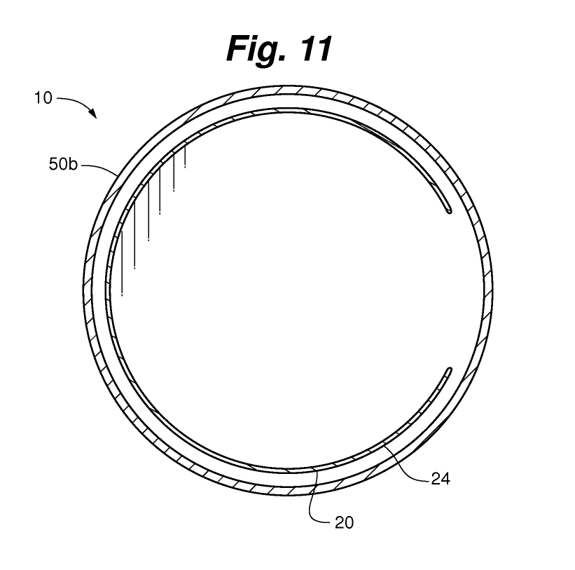

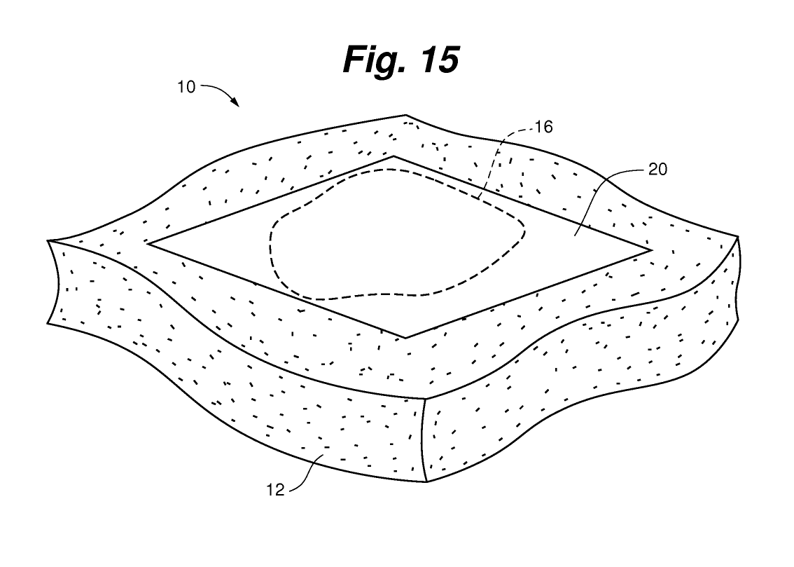

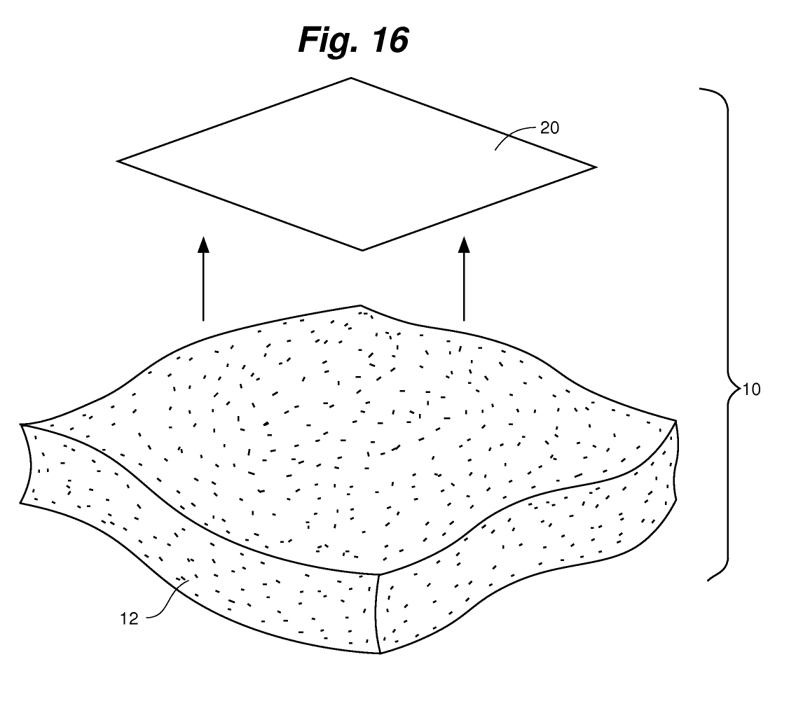

Turning now descriptively to the drawings, in which similar reference characters denote similar elements throughout the several views, FIGS. 1 through 16 illustrate a disinfectant system 10, which comprises a light source 14 for producing ultraviolet light, a fluid source 15 having a mass of organic contaminants 16 within and a carrier 20 comprising a substrate 21 and a photocatalyst 22. The photocatalyst 22 is evenly distributed throughout the substrate 21 so a treatment surface 24 of the carrier 20 is continually exposed to the fluid source 15 and the ultraviolet light as the substrate 21 degrades and substrate 21 is comprised of an electrically non conductive material. The treatment surface 24 is positioned at least partially within the fluid source 15 and wherein the ultraviolet light is focused upon the treatment surface 24 for oxidizing the mass of organic contaminants 16 within the fluid source 15. The carrier 20 may be used for various purposes, such as for disinfecting drinking water, ground surfaces 12, table surfaces and other contaminated objects. The carrier 20 may also be supported in various frames 30 or support structures.

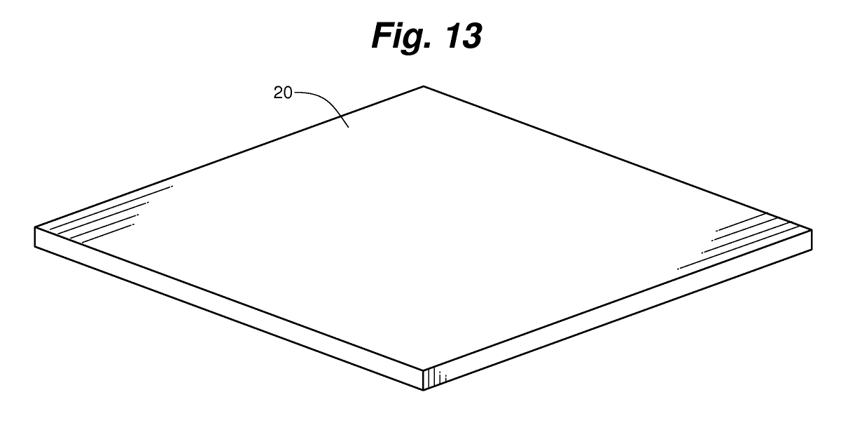

The fluid source 15 may refer to various types of fluids, such as a fluid source in a liquid state (e.g. water, etc.), a fluid source in a gaseous state (e.g. air), or a combination. For example, the liquid state may come into use when the carrier 20 is used within a reservoir structure 50a, 50b, 50c, such as a livestock tank as illustrated in FIGS. 1 through 7. The gaseous state of the fluid source 15 may come into use when the carrier 20 is used as a cutting board and thus disinfecting contaminants 16 upon the cutting board surface of the carrier 20 are within the surrounding air as illustrated in FIG. 13.

The light source 14 may also refer to various types of lights, such as a light source comprised of the sun, a light source comprised of ultraviolet light bulbs, or other ambient light sources. It is appreciated a partially obstructed light source 14 may also be used with the carrier 20. The ultraviolet light produces highly reactive forms of oxygen (oxygen free radicals and hydrogen peroxides) in the oxygenated fluid source 15 contribute in the destruction process of the microorganisms or organic contaminants 16 into oxidized particles 17.

The carrier 20 is used to oxidize the organic contaminants 16 within the fluid source 15 through a photocatalytic reaction between the carrier 20, ultraviolet light and the fluid source 15, wherein the fluid source 15 includes hydrogen elements and oxygen elements. The carrier 20 induces a chemical reaction to form a low amount of hydrogen peroxide to break down the contaminants 16 into oxidized particles 17 and thus effectively disinfect the fluid source 15 with the hydrogen peroxide. The carrier 20 may take the form of various shapes and configurations to fit within various size frames 30, other reservoir structures 50a, 50b, 50c, or be placed upon the ground surface 12 or various other objects as desired, whatever location has the need to disinfect or decontaminate. The carrier 20 is also substantially inert in the carrier 20 does not move during the chemical reaction, except the slight degrading of the substrate 21. The carrier 20 itself also can be comprised of a buoyant structure to float so the carrier may be placed within various fluid sources 15 and efficiently oxidize contaminants 16 near the surface of the fluid source 15.

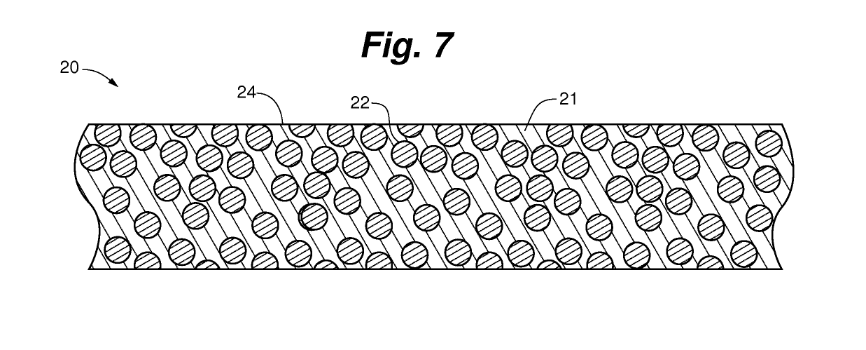

In the preferred embodiment, the carrier 20 is comprised of a substrate 21 and a photocatalyst 22 material coated within. The treatment surface 24 includes the portion of the carrier which has the photocatalyst 22 mixed with the substrate 21. The treatment surface 24 and photocatalyst 22 can be distributed evenly throughout the entire substrate 21 and thus entire carrier 20 as illustrated in FIG. 7. However, in alternate embodiments, the treatment surface 23 may be instead along the perimeter walls of openings extending through the carrier 20 (in the mesh shape), upon a top surface, a bottom surface, or portions thereof. The treatment surface 24 may simply be a small portion of the substrate 21 or carrier 20, of which contacts the fluid source 15 and receives the ultraviolet light from the light source 14. The substrate 21 may also be comprised of a permeable and absorbent structure so the contaminants 16 can travel within the carrier 20 to be oxidized within. It is appreciated various combinations of the above described, as well as other combinations, may also be used to combine the photocatalyst 22 with the substrate 21.

The substrate 21 is can be comprised of an electrically non conductive material, such as a plastic, which includes rubber, polystyrene, polymers, nylon, polyethylene, acrylic or various other types of plastic or non conductive materials and combinations of the various materials (e.g. substrate 21 comprised of rubber and polyethylene). The substrate 21 may also be absorbable to digest the contaminants 16 for the chemical reaction to take place. The use of a non conductive material, such as plastic, is important to provide an economic, variable product is easy to manufacture in various sizes, shapes and forms. The use of a plastic substrate 21 also provides a low melting temperature which helps to induce the chemical reaction and thus provide for a more efficient self disinfecting treatment surface 24.

The substrate 21 is pigmented with the photocatalyst 22 which can be comprised of titanium dioxide and has properties to induce a chemical reaction when exposed to ultraviolet light rays from the light source 14. The photocatalyst 22 further can be comprised of titanium dioxide in the anatase crystalline form rather than its rutile form. After the pigmentation melt process the substrate 21 including the photocatalyst 22 can be extruded in various forms whose surfaces 24 are photocatalytic in the oxidation of oxygenated water (e.g. fluid source 15) to hydrogen peroxide.

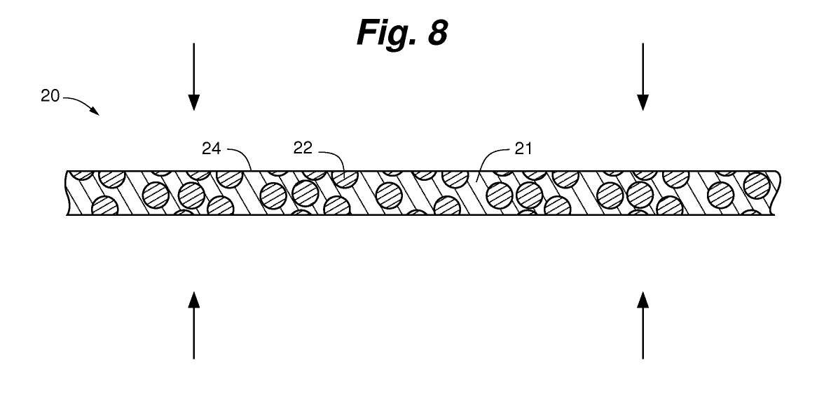

The photocatalyst 22 is comprised of an absorbing substance to be able to absorb the ultraviolet light. When receiving the ultraviolet light the photocatalyst 22 is able to oxidize the organic contaminants 16 to essentially self-disinfect the fluid source 15 or other type of surface or object. The treatment surface 24 extends throughout the carrier 20 and thus is continually exposed as the substrate 21 degrades away from the chemical reaction of the oxygen from the fluid source 15 and the ultraviolet light from the light source 14 to form hydrogen peroxide to break down the contaminants 16 into oxidized particles 17 as illustrated in FIG. 8.

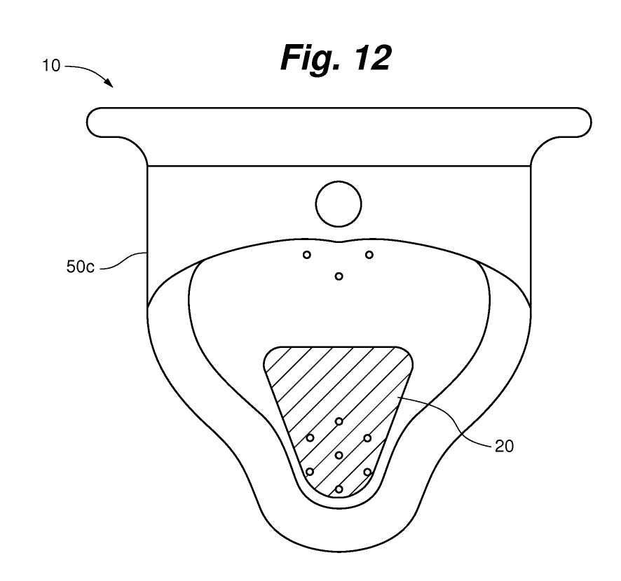

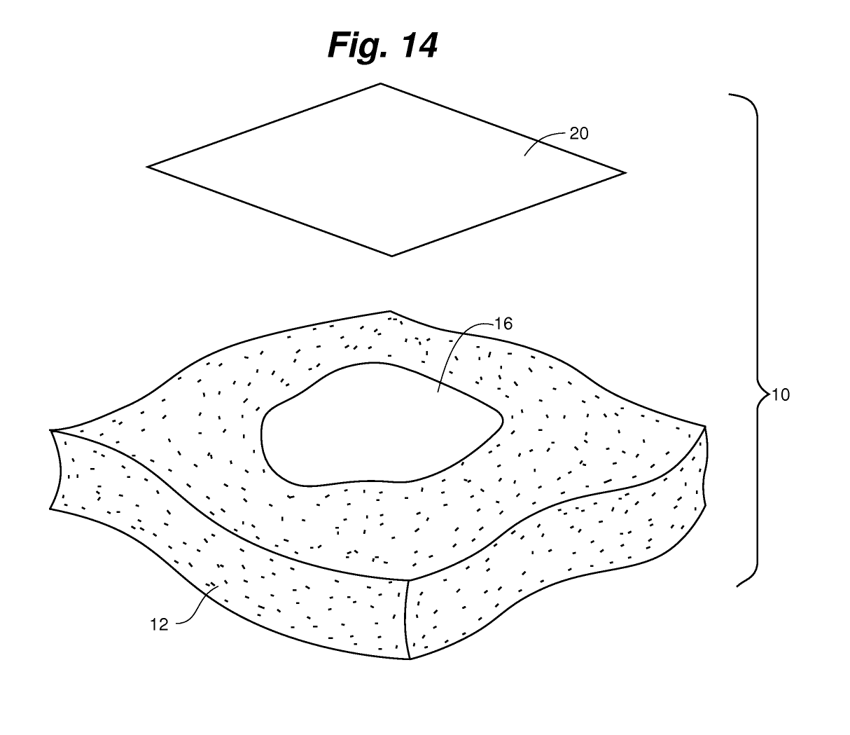

In one embodiment, the carrier 20 is formed into a mesh structure. The mesh structure allows the fluid source 15 to pass through while disinfecting the fluid source 15 by oxidizing the contaminants 16 therein. The mesh carrier 20 may be placed in various locations. One embodiment shows the mesh carrier 20 within the frame 30 for being positioned within a livestock tank as illustrated in FIGS. 1 through 7, another embodiment shows the carrier 20 positioned in a plastic drinking container to disinfect the water therein as illustrated in FIGS. 1 10 and 11, another embodiment shows the mesh carrier 20 positioned within a urinal over the drainage area to disinfect the urinal as illustrated in FIG. 12, and another embodiment shows the mesh carrier 20 positioned upon a ground surface 12 to oxidize and digest an oil spill area as illustrated in FIGS. 14 through 16. Various other sources may be used with the mesh carrier 20 other than those described. As appreciated, the mesh carrier 20 may be formed in various shapes and sizes.

When positioned around the float 40 of the frame 30, in one embodiment of the present invention, which will subsequently be described, the carrier 20 may include one or more openings 26 extending therethrough. The carrier 20 may also be secured to the frame 30 or other structure through the use of fasteners 27, such as but not limited to bolts.

In another embodiment of the carrier 20, the carrier 20 is formed into a cutting board configuration as illustrated in FIG. 13. Since the carrier 20 substrate 21 is comprised of a plastic, the carrier 20 is often molded into its final solid shape. In the case of the cutting board configuration, the carrier 20 is molded into a rectangular or other shaped cutting board. The photocatalyst 22 coating upon the substrate 21 of the carrier 20 is thus able to disinfect the cutting board surface (i.e. treatment surface 24) to keep the cutting board surface sterile or near sterile and provide a healthier atmosphere in which to serve and prepare food.

In one embodiment of the present invention, the frame 30 is used to support the carrier 20. The frame 30 is generally comprised of a rectangular or square shaped structure; however it is appreciated other shapes may be appreciated. The frame 30 is configured to be positioned within a reservoir structure 50a comprised of a livestock tank commonly used to hold water for livestock to drink. The carrier 20 thus in the frame 30 serves to disinfect the water within the reservoir structure 50a thus providing a clean contaminant free water for the livestock.

In an embodiment, the frame 30 includes a lower wall 31 including a plurality of inlets 32 spaced around an inner perimeter and a lower receiver opening 33 generally extending through a central portion of the lower wall 31. Sidewalls 39 vertically extend from the outer perimeter of the lower wall 31 and an upper wall 35 is attached to the upper end of the sidewalls 39, thus vertically offsetting the upper wall 35 with respect to the lower wall 31. The upper wall 35 includes a plurality of outlets 36 to substantially align with the inlets 32 of the lower wall 31 and an upper receiver opening 37 also can be near a center of the upper wall 35 similar to the lower receiver opening 33.

The carrier 20 is can be affixed to the upper surface of the lower wall 31 and thus within a cavity 38 defined between the upper wall 35 and the lower wall 31. The cavity 38 is can be substantially larger in height than the carrier 20 to allow room for the oxidized particles 17 to escape through the outlets 36 of the upper wall 35. The carrier 20 may be affixed to the lower wall 31 in various manners, such as through the use of the fasteners 27 (e.g. bolts. etc.) or other securing mechanisms.

The treatment surface 24 of the carrier 20 is can be positioned directly over the inlets 32 so the contaminants 16 can easily engage the treatment surface 24 and thus be oxidized by the photocatalytic reaction. A plurality of inlets 32 ma extend through the lower wall 31 so the fluid source 15 having the contaminants 16 may engage the carrier 20 in a plurality of different locations. Once the contaminants 16 are oxidized by the photocatalytic reaction, the oxidized particles 17 can escape the cavity 38 through the outlets 36 of the upper wall 35.

The frame 30 and at least the upper wall 35 is also comprised of a transparent configuration to allow the ultraviolet light from the light source 14 to pass through and be focused upon the treatment surface 24 of the carrier 20. The upper wall 35 also serves another purpose, besides providing support for the frame 30, which is to protect the carrier 20 by preventing the livestock or foreign particles from engaging or contacting the carrier 20. The upper wall 35 and thus sidewalls 39 thus extend over and surround the entire carrier 20 besides the portion of the carrier 20 is accessible through the inlets 32 and outlets 36. However, the inlets 32 and the outlets 36 are substantially small, wherein only contaminants 16 within the fluid source 15 need to pass through the inlets 32 and oxidized particles 17 need to pass through the outlets 36.

A float 40 is connected to the frame 30 to provide buoyancy for the frame 30 so the frame 30 can stay afloat within the fluid source 15 of the reservoir structure 50a. In the preferred embodiment, the float 40 provides just enough buoyancy so the carrier 20 is submerged within the fluid source 15 yet the upper wall 35 is positioned above the surface of the fluid source 15. The float 40 may be comprised of various types of foam or other floatable structures. The float 40 is tightly positioned within the lower receiver opening 33 and extends upwards to engage the lower surface of the upper wall 35.

In an alternate embodiment, the float 40 is comprised of a heating source, which is primarily used to heat. The fluid source 15 within the reservoir structure 50a during cold periods to prevent the fluid source 15 from freezing. Thus, the float 40 serves dual purposes of keeping the frame 30 afloat and heating the fluid source 15 to prevent freezing. In this embodiment, the upper receiver opening 37 is used, wherein the cord 41 from the heater configuration of the float 40 extends through the upper receiver opening 37 and the cord 31 includes a plug 42 which is electrically connected to an electrical socket to operate the heater comprised float 40.

As discussed previously, the reservoir structure 50a is can be used to hold the fluid source 15 for livestock, wherein the fluid source 15 is water. However, the reservoir structure may take the form of various other embodiments, such as a plastic water bottle 50b, wherein the frame 30 may be omitted and the carrier 20 is simply wrapped around the inside perimeter of the bottle casing. Another embodiment shows the reservoir structure 50c comprised of a toilet or urinal configuration and the carrier 20 simply positioned over the drain opening to disinfect fluid sources come into contact with the carrier 20 within the urinal or toilet. Various other embodiments as discussed (e.g. cutting board, carrier 20 to clean up spills on a ground surface 12 such as an oil spill, etc.) may be used with the carrier 20. It. is appreciated the carrier 20 may be used for further embodiments all of which require the disinfection of a fluid source.

In use, the frame 30 including the carrier 20 is positioned within the fluid source 15 of the reservoir structure 50a so the lower wall 31 faces downward. The float 40 allows the carrier 20 and lower wall 31 to sink within the water either partially or wholly while keeping the upper wall 35 above the water surface so the oxidized particles 17 can more easily escape.

As the fluid source 15 including the organic contaminants 16 contacts the treatment surface 24, the oxygen from the fluid source 15 and the ultraviolet light from the light source 14 induce a chemical reaction with the photocatalyst 22 to form an antibacterial (e.g. hydrogen peroxide). The antibacterial generated from the photocatalytic reaction thus oxidizes the fluid source 15 including the contaminants 16 to disinfect the fluid source 15.

The carrier 20 continues to operate as long as the carrier 20 is positioned at least partially within the fluid source 15 containing oxygen. As the chemical reaction takes place, the substrate 21 slowly degrades. However, since the photocatalyst 22 is positioned evenly throughout the substrate 21 the carrier 20 continually exposes a treatment surface 24 including the photocatalyst 22 and the substrate 21 to the fluid source 15 and the light source 14.
110) SolaCleanse Mexico depicts a complete water purification system given a tank source.
111) Barrel, cover, and generator assembly.
112) Vent provides air to the injector for aeration of the incoming flow.
113) Acid reservoir provides acid to the injector for acidification of the incoming flow
114) Check valve allows incoming flow while stopping backflow to the reservoir.
115) Solenoid valve regulates flow of acid to injector. Controlled by pH/ORP controller
116) pH/ORP controller collects data from sensors and controls the solenoid valve. Computer interface is optional.
117) Injector throttles flow from tank for suction.
211) Barrel, cover and generator assembly.
210) Tank lid keeps water in barrel clean and allows sunlight in.
211) Hydrogen peroxide generator floats on surface of water and produces hydrogen peroxide.
212) Barrel is lined with Mylar to reflect incoming sunlight to the generator surface.
Step 1) Tank begins filling.
Step 2) Water proceeds out at a point near its bottom. It flows past the check valve.
Step 3) Past the check valve it encounters the injector where acid from the acid reservoir enters the stream along with air from the vent. It then encounters the first pH probe, which with the help of the controller meters the flow acid via a pinch valve, which is under the control of the controller.
Step 4) The water then enters the barrel and begins to support the float on the generator. Light entering at the top of the barrel irradiates the upper surface of the generator where hydrogen ion and free oxygen unite to produce hydrogen peroxide. The peroxide then begins to kill microorganisms, any unused hydrogen peroxide is returned to its constituent part, water and free oxygen due to the anti oxidant properties of the citrate ion. The rise in pH due to consumption of the hydrogen ions causes acetates of metal ions to precipitate out of solution.
Step 5) pH changes at the bottom of the barrel allows the controller to demand acid from the acid reservoir via the pinch valve. ORP is also monitored to assure proper disinfection of the water in the barrel.
Step 6) Flow then continues on demand from the user, through a filter to the user.
   Note: When citric acid is used. Excess citric acid in trace amounts is delivered to the user giving the final product a slight sour taste. Similar to rainwater, which if used as the stock water obviates the need for acidification. Some filtration will be necessary with the use of citric acid.
Step 1) User fills clear container containing SolaCleanse Grid with questionable water.
Step 2) User adds citric acid tablet and exposes container to sunlight.
Step 3) User allows container to receive sunlight until the water gets cloudy.
Step 4) User filters now disinfected water. The water is now ready to drink.

Thus, embodiments of the EMBEDDED PHOTOCATALYST FOR HYDROGEN PEROXIDE PRODUCTION are disclosed. One skilled in the art will appreciate the present teachings can be practiced with embodiments other than those disclosed. The disclosed embodiments are presented for functions of illustration and not limitation, and the present teachings are limited only by the claims follow.

The invention claimed is:

1. A disinfectant system, comprising:
a light source for producing ultraviolet light;
a fluid source having a mass of organic contaminants within; and
a carrier comprising a substrate and a photocatalyst;
wherein said, photocatalyst is evenly distributed throughout said substrate so a treatment surface of said carrier having said photocatalyst and said substrate is continually exposed to as fluid source and said ultraviolet light as said substrate degrades;
wherein said substrate is comprised of an electrically non conductive material;
wherein said treatment surface is positioned at least within said fluid source and wherein said ultraviolet light is focused upon said treatment surface for oxidizing said mass of organic contaminants within said fluid source

2. The disinfectant system of claim 1, wherein said substrate is comprised of a plastic.

3. The disinfectant system of claim 1, wherein said substrate is comprised of a rubber.

4. The disinfectant system of claim 1, wherein said photocatalyst is comprised of titanium dioxide.

5. The disinfectant system of claim 4, wherein said titanium dioxide is in an anatase form.

6. The disinfectant system of claim 1, wherein said substrate is comprised of a plastic and wherein said photocatalyst is comprised of titanium dioxide in an anatase form.

7. The disinfectant system of claim 1, wherein said carrier is comprised of a buoyant structure.

8. The disinfectant system of claim 1, including:
a frame positioned within said fluid source; and
a float connected to said frame, wherein said float maintains said frame at least partially buoyant within said fluid source;
wherein said carrier is connected to said frame. so said carrier is suspended at least partially below a fluid source of said fluid source.

9. The disinfectant system of claim 8, wherein said frame includes a lower wall including a plurality of inlets for receiving said mass of organic contaminants and an upper wall including a plurality of outlets for releasing said oxidized mass of organic contaminants, wherein said upper wall is spaced apart from said lower wall.

10. The disinfectant system of claim 9, wherein said carrier is connected to said lower wall between said lower wall and said upper wall.

11. The disinfectant system of claim 1, wherein said carrier is positioned within a water bottle.

12. The disinfectant system of claim 1, wherein said carrier is positioned within a urinal.

13. The disinfectant system of claim 1, wherein said carrier is comprised of a mesh shaped structure.

14. The disinfectant system of claim 1, wherein said carrier is comprised of a cutting board structure.

15. A disinfectant system, comprising:
a frame having a lower wall including at least one inlet and an upper wall including at least one outlet, wherein said lower wall is vertically offset with respect to said upper wall;
wherein at least said. upper wall of said frame is transparent;
a float connected to said frame for providing buoyancy to said frame; and
a carrier connected to said lower wall between, said lower wall and said upper wall, wherein said upper wall substantially surrounds an upper surface of said carrier;
wherein said carrier induces a photocatalytic reaction;
wherein said carrier is in fluid communication with said at least one inlet for receiving a mass of organic contaminants from a fluid source;
wherein said carrier is in fluid communication with said at least one outlet for releasing a mass of oxidized particles generated during said photocatalytic reaction.

16. The disinfectant system of claim 15, wherein said carrier is comprised of a substrate and a photocatalyst, wherein said photocatalyst is evenly distributed throughout said substrate.

17. The disinfectant system of claim 16, wherein said substrate is comprised of a plastic and wherein said photocatalyst is comprised of titanium dioxide in an anatase form.

18. The disinfectant system of claim 15, wherein said float is comprised of a heating source.

19. The disinfectant system of claim 15, wherein said carrier is comprised of a mesh shaped structure.
